(19) United States
(12) Patent Application Publication
(10) Pub. No.: US 2024/0391874 A1
(43) Pub. Date: Nov. 28, 2024

(54) NOVEL, SAFE, ECONOMICAL AND AN EFFICIENT PROCESS FOR PREPARATION OF 3-FLUORO-2-NITRO PYRIDINE OF FORMULA I

(71) Applicant: AASTRID LIFE SCIENCES PVT. LTD., NAVI MUMBAI (IN)

(72) Inventors: VIKRAM JAGTAP, NAVI MUMBAI (IN); MAHESH TELI, NAVI MUMBAI (IN); SAMBHAJI KUMBHAR, NAVI MUMBAI (IN); YUSUF DARBAR, NAVI MUMBAI (IN); GANESH DESHMUKH, NAVI MUMBAI (IN); RAVINDRA JAGTAP, NAVI MUMBAI (IN)

(21) Appl. No.: 18/259,487

(22) PCT Filed: Aug. 24, 2022
(86) PCT No.: PCT/IN2022/050754

§ 371 Date: Jun. 27, 2023

(30) Foreign Application Priority Data
Dec. 20, 2021 (IN) 202121059348

(51) Int. Cl. C07D 213/76

(52) U.S. Cl. CPC C07D 213/76

(57) ABSTRACT

A novel, safe and an efficient high yielding process for preparation of substantially pure 3-fluoro-2-nitro pyridine of formula I, devoid of hazards like sudden exotherm, decomposition of the desired intermediate, explosion, charring etc. comprising salt decomposition of fluoroborate salt of formula II into 3-fluoro-2-nitro pyridine of formula I. The process of the present characterized by the fact that fluoroborate salt of formula II is added in lots in a high boiling solvent like maintaining the temperature in the range of 95-100° C. thereby by avoiding all the hazards and providing a novel and safe process.

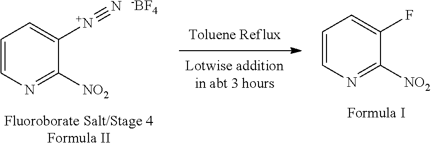

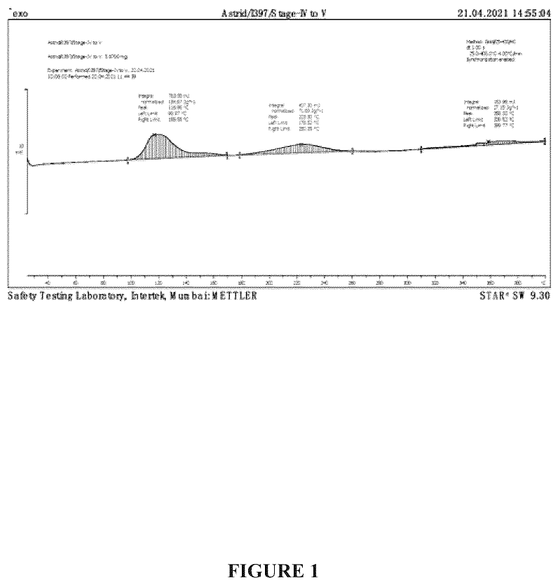

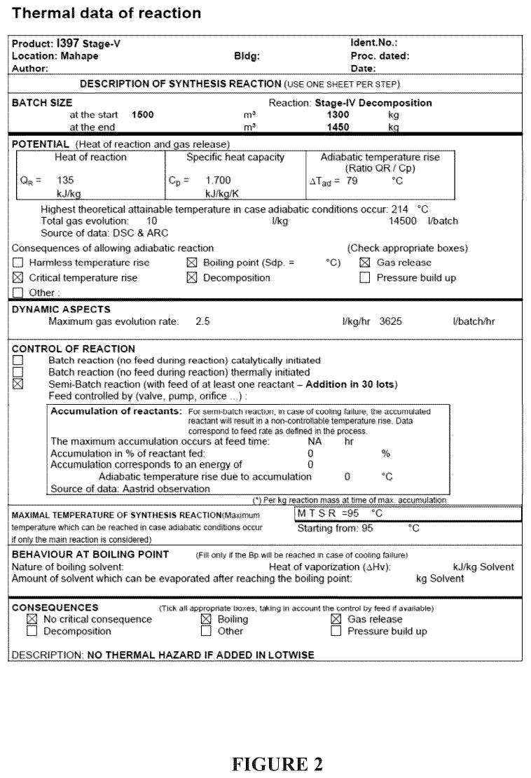

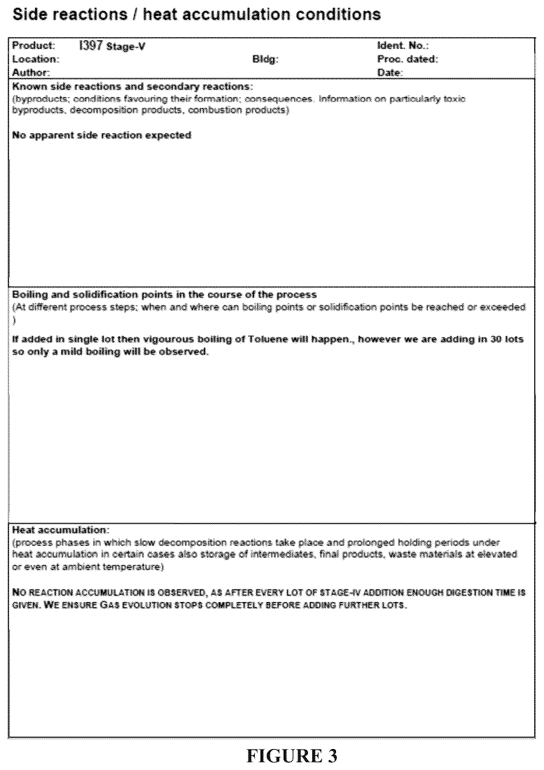

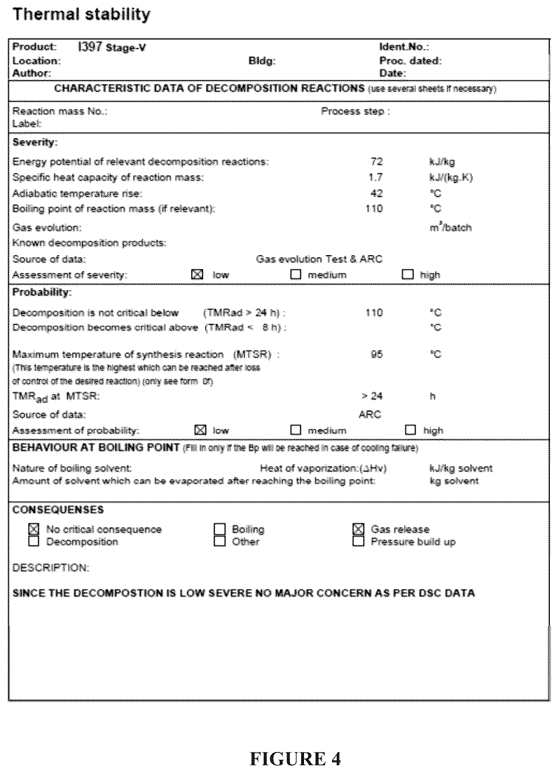

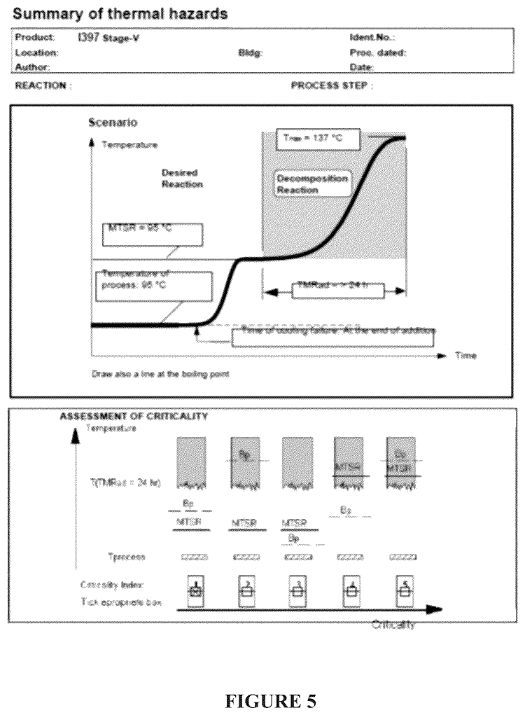

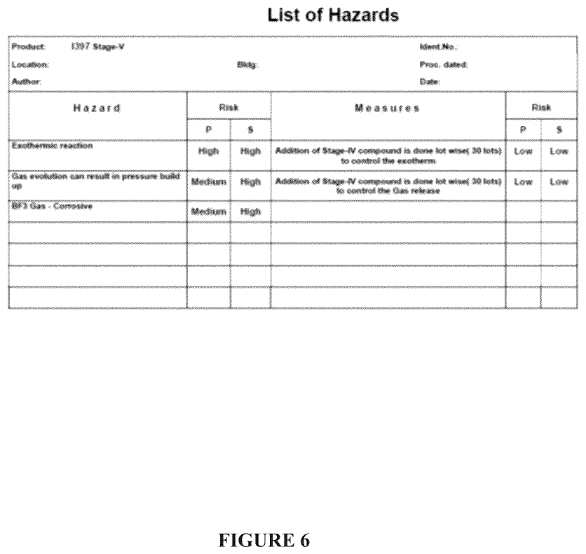

### FIELD OF TECHNOLOGY

Disclosed herein is a safe, economical and an efficient scalable process for the preparation of substantially pure pharmaceutical intermediate3-fluoro-2-nitro pyridine of formula I in high yield comprising diazotization of 3-amino-2-nitropyridine of formula III with aqueous solution of sodium nitrite in presence of fluoroboric acid to obtain fluoroborate salt of formula II followed by thermal decomposition of the said salt of Formula II to obtain the 3-Fluoro-2-nitro pyridine of formula I as illustrated herein below in SCHEME A:

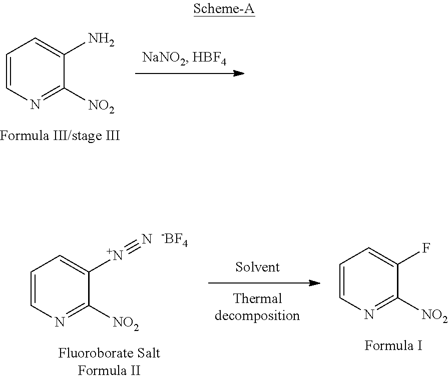

In particular present invention pertains to an improved process for the preparation of 3-fluoro-2-nitro pyridine of formula I comprising thermal decomposition of an intermediate of formula II. The compound of formula II is a fluoroborate salt having a nitro group, hence associated with risky parameters when taken for the thermal decomposition to yield desired compound 3-fluoro-2-nitro pyridine of formula I as illustrated herein below in SCHEME-I.

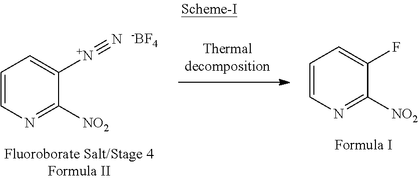

Disclosed herein is an efficient and safe process for the decomposition of compound of Formula II avoiding all the unsafe shortcomings of the prior art processes and produce a substantially pure compound of Formula I in high yield. The process of the present invention is characterized by the inventive feature comprising a lot wise addition of fluoroborate salt of formula II into a high boiling solvent in about three to four hours; maintaining the temperature of the reaction mass in the range of about 85-95° C. followed by the refluxing till the completion of the reaction. This modified mode of working not only avoids all the hazards like explosion, charring, decomposition of the desired product of formula I to undesired impurity formation etc. but also provides higher yield with better purity than that of the prior art processes.

The entire general synthetic scheme for the preparation of 3-fluoro-2-nitro pyridine of formula I is illustrated herein below in SCHEME II

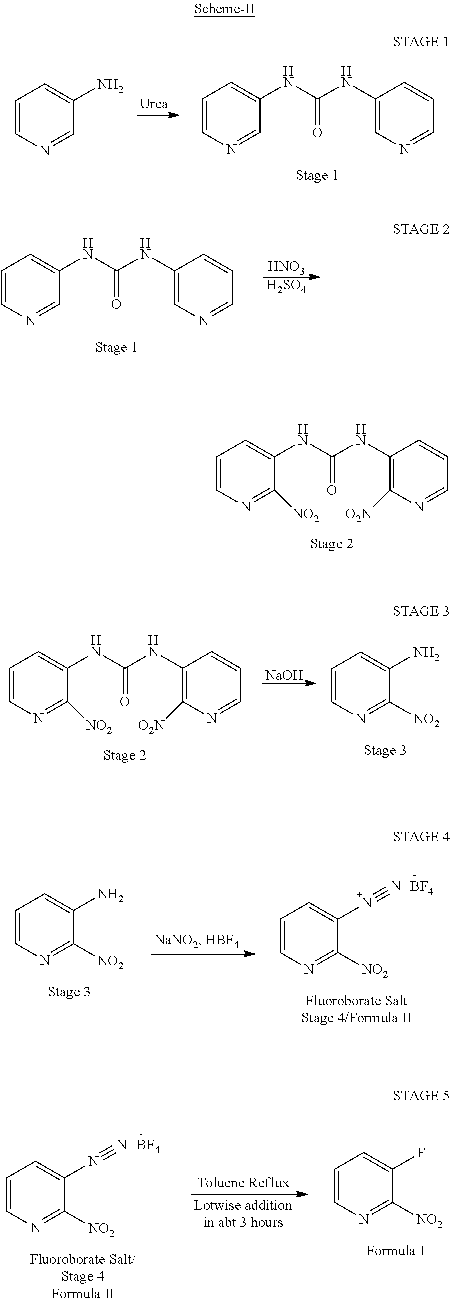

Wherein; the most interesting feature of the present invention pertains to changes occurring during stage 5 comprising safe, green and economical transit of compound of formula II into compound of formula I.

### BACKGROUND OF THE INVENTION

3-fluoro-2-nitro pyridine is an important pharmaceutical intermediate required for the preparation of API Crizotinib, an anti-cancer medication acting as an ALK and ROS1 inhibitor. However; synthesis of 3-fluoro-2-nitro pyridine requires Fluoroborate salt of formula II as key raw material which on decomposition yields 3-fluoro-2-nitro pyridine of formula I. Fluoroborate compound of formula II is a nitro compound hence the said decomposition process remains associated with hazards like explosion, decomposition with undesired impurity formation and charring.

The processes disclosed in the prior art comprising decomposition of Formula II is associated with shortcomings viz exotherm, charring, decomposition of the product (thereby generating undesired impurities) and hence possibility of explosion.

WO2008109613 discloses a process for the preparation of 3-fluoro-2-nitro pyridine comprising dropwise addition of sodium nitrite into a stirred mixture of 3-amino-2-nitropyridine in 34% fluoroboric acid. Temperature is maintained between −8° C. to −2° C. during the addition. The reaction mixture is stirred at −2 to −8 temperature for about 30 minutes. The suspension is filtered and the solid obtained is washed further with 34% fluoroboric acid followed by ether and dried under vacuum to provide a fluoroborate salt of formula II. However; it is to be noted that process comprises single lot addition in toluene followed by reflux for about 4 hours. This single lot addition increases of exotherm, when immediate rise of temperature takes place thereby making the process very dangerous and unviable at commercial scale.

Drawback associated with this particular process is that a single lot addition of fluoroborate salt of formula II in toluene (1:15 w/v) at 30-35° C., results in sudden exotherm when the temperature shoots up from 65 to 100° C. with a lot of foaming even at a small scale as observed by the inventors of the present invention. Foaming and evolution of gases like inert nitrogen and corrosive boron trifluoride occurring during the processing results in to the spillage of the reaction mass thereby developing pressure and also a risk of catching fire making process unsafe. This unsafe mode of at once one lot addition can be a cause of explosion which makes the process very dangerous and unviable at commercial scale. Apart from this, degradation of product so obtained results in low yield and high impurity and making the process cumbersome to remove impurities. Further, 3-fluoro-2-nitro pyridine (2.8% yields) is obtained as brown oil thereby making the said process industrially inefficient.

WO2006044355 discloses drop wise addition of aqueous solution of sodium nitrite to a stirred mixture of 2-nitropyridine-3-amine in 34% fluoroboric acid and during addition the temperature is maintained between −8° C. to −2° C. After 0.5 h, the suspension was filtered and the solid washed with 34% fluoroboric acid followed by washing with ether and dried at room temperature under high vacuum for 12 h to give an orange brown solid of the fluoroborate salt of formula II. The dry solid as such is decomposed by heating to 120° C. After decomposition the remaining oil is treated with a solution of 10% sodium hydrogen carbonate and the mixture is extracted with dichloromethane. The combined extracts are dried over sodium sulfate, filtered and the solvent removed over under reduced pressure to yield the title compound 3-fluoro-2-nitropyridine as a pale yellow solid.

Drawback associated with this process is that it comprises decomposition of fluoroborate salt of formula II in solid state at 120° C. which poses a high risk of explosion (being a nitro compound) along with dense fume formation associated with the liberation of inert nitrogen and corrosive boron trifluoride gas thereby developing high pressure. Further to this, oil so obtained after decomposition is further treated with bicarbonate followed by its extractions; solvent is removed by vacuum distillation to get the desired product of formula I as pale yellow solid. The said process does not report the yield and purity of the desired product.

Another major drawback of prior art processes as disclosed herein above is that these are conducted only on milligram to 20 gram scale without any indication of its scalability to industrial scale.

As evident from prior art processes disclosed herein above either comprises addition of fluoroborate salt in a single lot followed by reflux in toluene for 4 hours or involves the decomposition of fluoroborate salt in dry state as such. Both the processes are associated with dangers like sudden exotherm, immediate rise in temperature along with uncontrollable frothing/foaming which causes the spillage of the solvent, toxic and dense fumes formation making the process very dangerous and unviable at commercial scale. This also results in degradation of product resulting in low yield and low purity and making the process cumbersome to remove impurities.

### Characterization Techniques:

Looking at various drawbacks as mentioned herein above, it was essential to study the thermo chemistry of the reaction comprising decomposition of compound of formula II yielding the compound of formula I represented in SCHEME-I. In view of above there is a dire need to overcome the shortcomings such as exotherm characteristics, solvent spillage, foaming, pressure development, evolution of dangerous gasses, decomposition of subject matter product to form undesired impurities, charring in the prior art process for the preparation of compound of Formula I for which there is no teaching or motivation to a person skilled in the art. Inventors of the present invention have studied various parameters like differential scanning colorimetry (DSC) and accelerated reaction colorimetry (ARC) associated with the hazards during the stage 5 (involving decomposition) i.e. transit of fluoroborate compound of formula II into compound of formula I as illustrated in SCHEME-1. As per DSC, it was found that during this transit, multiple exotherms with liberation of thermal energy were observed thereby confirming exotherms and temperature shoot up.

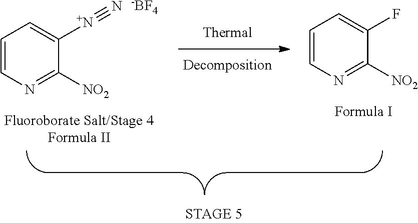

DSC of stage 5 was studied on the scale of 0.867 gram of fluoroborate salt of formula II (stage 4 material). DSC reveals that these multiple exotherms shows 1^{st} exotherm onset at 97° C. with evolution of heat 135 J/g of sample, 2^{nd} exotherm onset at 179° C. with evolution of heat 72 J/g of sample and 3^{rd} partial exotherm onset at 310° C. with evolution of heat 27 J/g of sample. In simple words said conversion is associated with the liberation of thermal energy with rise in temperature thereby indicating a warning alarm and dire need of safety measures to be taken.

Inventors of present invention critically analyzed the DSC data of the prior art processes and found that there is a need to develop an innovative and improved hazards free process. Inventors of the present invention had generated the DSC data of the process of prior art processes and based on its analysis disclosed herein a novel and innovative process for the decomposition of fluoroborate compound of formula II into desired compound of formula I comprising lot wise addition of compound of formula II into a solvent preferably toluene at a temperature about 85-90° C. and temperature of the reaction mixture rises to about 96° C. Remaining lots are added in similar way by providing the proper digestion period during which rise in temperature and gas evolution remains under control thereby all the associated hazards are mitigated and the process becomes green and industrially safe.

Inventors of the present invention have followed ditto process disclosed in the prior art and as per their finding, the results of same are very much unfavorable. As per their observations, when said decomposition of compound of Formula II is carried out comprising a single lot addition on the scale of 100 gm fluoroborate salt of formula II in toluene (1:15 w/v) followed by gradual increase in the temperature to attain the reflux results in vigorous foaming/frothing & temperature shoots from 65° C. to 100° C. (exotherm) with gas evolution. If gas evolution and foaming/frothing are not controlled, it pressurizes the reaction and result into spillage of the solvent, decomposition/charring of the product and possibility of explosion. On the other hand when decomposition of fluoroborate salt of formula II is conducted in dry state, temperature shoots from 45 to 120° C. with dense fumigation with gas evolution and the material gets charred. Side products are inert nitrogen gas and corrosive boron trifluoride gas (BF3).

In addition to DSC, accelerating rate calorimetry (ARC) was also performed to identify the safety aspects of the process of the present invention which deals with evaluation of hazard prediction for reactive chemicals. In the present study of decomposition of stage 5 (compound of Formula II into compound of Formula I to I) is evaluated, wherein, decomposition of compound of formula II comprises the addition in about 30 lots in a suitable solvent maintaining temperature 85-95° C. is safe way of decomposition of compound of formula II yielding the compound of formula I. Finding of the current study involving lot wise addition reveals that the “maximum temperature of the synthetic reaction (MTSR)” reaches to 95° C. indicating no thermal hazard if added in small lots with no apparent side reactions as well. This mode is further characterized by the fact that no heat accumulation is observed as after each lot addition as enough digestion time is provided which also results with controlled gas evolution; thereby making the process at low risk and thus makes it industrially feasible and safe.

As per the experience of the inventors of the present invention while performing the ditto study of prior art processes, it was decided by them to investigate the thermal study of the reaction of interest associated with that of stage 5 including DSC, ARC etc. as defined herein below:

### Defenition of the Technical Terms (Characterizationtechniques) Used:

THERMOCHEMISTRY: Thermochemistry is the study of the heat energy which is associated with chemical reactions and/or physical transformations. A reaction may release or absorb energy and a phase change such as in melting and boiling.

DIFFERENTIAL SCANNING COLORIMETRY (DSC): Differential Scanning calorimetry (DSC) is a thermal analysis technique in which the heat flow into or out of a sample is measured as a function of temperature or time, while the sample is exposed to a controlled temperature program. Study of DSC indicates how physical properties of a sample change, along with temperature against time. In other words, the device is a thermal analysis instrument that determines the temperature and heat flow associated with material transitions as a function of time and temperature.

ACCELERATING RATE CALORIMETRY: Accelerating rate calorimetry is a common tool used in thermal stability evaluation of hazardous materials to provide self-heat rate and pressure data that are used to model the kinetics of a reaction.

It is used to obtain information about the thermal behavior of reactions, reaction onset temperature of reaction, heat release rate, pressure rise rate and amount of condensable vapor and non-condensable gas generated. It is also used for safety/performance evaluation of explosives and propellants.

TECHNICAL PROBLEMS ASSOCIATED WITH THE PRIOR ART: Processes disclosed in the prior art are associated with the risks like explosion, decomposition of the product, low yield, nonviable on commercial scale. Additionally, the prior art processes are prone to the formation of by-products/impurities resulting from decomposition of compound of Formula I. One of the prior art process comprising dry distillation of fluoroborate salt of formula II into I also requires extra steps like multiple extractions, manual separations etc. thereby increasing the overall cost of the process making it uneconomical and nonviable on commercial scale. Furthermore, the decomposition products formed are inert nitrogen gas but hazardous and corrosive boron trifluoride (BF₃) gas. This all leads to poor yield, environmental hazard and inferior quality at intermediate stages as well as at the final product stage thereupon.

In view of above there is a dire need to find an effective solution that will provide a safe and efficient, high yielding process especially during the stage 5 comprising the decomposition of compound of formula II into compound of formula I.

TECHNICAL SOLUTION: The technical problem to be solved by the present invention is to overcome the above-mentioned deficiencies of prior art as mentioned herein before. Inventors of the present invention have provided a novel, green, economical and safe process for the preparation of 3-fluoro-2-nitro pyridine. Looking at the various problems associated with work-up and isolation of the desired product 3-fluoro-2-nitro pyridine there remains a need in the art to provide an efficient, safe, industrially scalable high yielding process for the preparation of 3-fluoro-2-nitro pyridine wherein substantially pure product is obtained in good yield without using cumbersome extraction process as disclosed in prior art process.

To avoid the risks associated with the prior art processes, inventors of the present invention have provided the option of the decomposition of fluoroborate salt comprising the lot wise addition of the said salt of Formula II in solvent maintaining the temperature in the range of about 85-95° C. followed by reflux till the completion of the reaction. There has not been any indication, teaching or motivation to a person skilled in the art from the prior art to consider lot wise addition of compound of formula II instead of adding whole quantity in a single lot thereby controlling excessive exothermic characteristic of the decomposition reaction of compound of formula II into compound of Formula I and avoiding formation of undesired impurities, charring and making the process green and safe to give substantially pure 3-fluoro-2-nitropyridine of Formula I in good yield.

The present invention provides technical solutions to overcome the drawbacks of the prior art processes specifically associated with the decomposition of fluoroborate salt of formula II into 3-Fluoro-2 nitro Pyridine of formula I thereby improving the overall process as represented in SCHEME-1. In other words, conversion of the compound of formula II into compound of formula I as illustrated in the SCHEME-1 and/or stage 5 is of major concern and to provide a technical solution is the most important task of the inventors of the present invention.

Advantageously, the process of the present invention results in the formation of the compound of Formula-I in a high yield and high purity compared to the prior art processes. Additionally, the process is a safe and eco-friendly avoiding hazard of the prior art processes. Hazardous and corrosive BF₃ gas evolved as flue gas is scrubbed in sodium hydroxide solution thereby producing non-hazardous boric acid sodium salt and sodium fluoroborate.

The “novelty” and “inventive feature” in the improved process is in providing the technical solution, which lies in the lot wise addition of the said fluoroborate salt of Formula II maintaining the temperature in the range of about 85-95° C., thereby avoiding the risk of sudden exotherm, uncontrollable foaming, dense fumes formation, explosion etc. thereby providing low risk or no risk high yielding industrially scalable and viable process for the preparation of compound of formula I.

### Industrial Advantages:

The present invention provides an efficient industrial safe process which provides 3-Fluoro-2-nitro pyridine with improved yield 30%. In particular, inventors of the present invention are getting yield in the range of 30 to 35% yield for conversion of fluoroborate salt into 3-Fluro-2 nitro pyridine. This is substantially higher than that of the prior art yield of 2.8%.

The loading quantity fluoroborate salt of stage 4 is done in small portions/lots to avoid decomposition which produces hazardous, lachrymatic, toxic, foaming/frothing, spillage of solvent thereby reduces all the risk associated with prior art processes.

Large scale batch size is possible to be taken making it commercially feasible.

### OBJECTS OF THE INVENTION

One of the major aspects of the present invention is to provide a novel and safe process for the preparation of compound of formula I comprising decomposition of fluoroborate salt of formula II by adding in multiple portions in a solvent by maintaining the temperature about 85-95° C. followed by reflux till the decomposition gets completed.

Another aspects of the present invention is to provide a novel and safe process for the preparation of compound of formula I comprising the diazotization of compound 3-amino-2-nitropyridine of formula III with aqueous solution of sodium nitrite in presence of fluoroboric acid resulting into the formation of fluoroborate salt of formula II followed by its thermal decomposition of compound of Formula II to obtain the 3-Fluoro-2-nitro pyridine of formula I.

One of the aspects of the present invention is to provide an equal balance between process and analytical parameters which worked side-by-side to develop safe, scalable, efficient and cost-effective chemical processes for the preparation of 3-Fluoro-2-nitro Pyridine of formula I. One of the aspects of the present invention is to analyze the analytical parameters and compare the safety measures of the present process Vs process of the present invention.

One of the aspects of the present invention is to provide novel, efficient, safe, industrially scalable, high yielding process for the preparation of substantially pure 3-fluoro-2-nitro pyridine with minimum or no risk.

One of the aspects of the present invention is to avoid hazards like explosion. decomposition of the required product and therefore associated risk of blast, frothing thereafter spillages of the solvent and hence risk of fire, charring of the product etc. are avoided.

One of the aspects of the present invention is to obtain the desired 3-fluoro-2-nitro pyridine with higher yield and high purity.

One of the aspects of the present invention is to provide an industrially scalable process involving safe mode especially of decomposition of fluoroborate salt of formula II resulting in to compound of formula I and/or stage 5 comprising lot wise additions.

One of the aspects of the present invention is to scrub the corrosive BF₃ gas in caustic solution thereby converting it into sodium borate salt.

One of the aspects of the present invention is to study analytical parameters like differential scanning calorimetry (DSC) to determine the temperature and heat flow associated with material transitions as a function of time and temperature especially w.r.t. stage 5 to evaluate the transit of compound of formula II into compound of formula I.

One of the aspects of the present invention is to study analytical parameters like accelerated rate calorimeter (ARC) which provides full adiabatic runaway information for both temperature and pressure events especially w.r.t. stage 5 to evaluate the transit of compound of formula IV in compound of formula V.

### SUMMARY OF THE INVENTION

Disclosed herein is a safe, novel improved process for the preparation of substantially pure in high yield 3-fluoro-2-nitro pyridine. The said innovative improved process ensures enhanced safety and eco-friendly norms. The present disclosure relates to novel, safe and efficient process for the synthesis of 3-Fluoro-2-nitro Pyridine which is applicable even on large scale making it commercially feasible.

The present invention further relates to an improved process for the conversion of fluoroborate salt of Formula II into desired product 3-Fluoro-2-nitro Pyridine of formula I, wherein fluoroborate salt of Formula II is added in multiple small portions/lots into a solvent in about 3-4 hours and maintaining the temperature at about 85-95° C. till the decomposition of salt of formula II followed by reflux till the reaction gets completed in high yield and purity.

Schematic representation for the preparation of 3-Fluoro-2-nitro Pyridine is as given herein below as SCHEME-I:

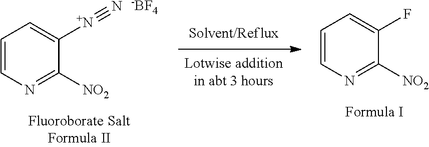

Analytical data obtained from DSC and ARC studies clearly indicate that the process of the present invention for the preparation of 3-Fluoro-2-nitro Pyridine of formula I comprising lot wise addition of the fluoroborate salt of formula II maintaining the temperature below 100° C. to obtain compound of formula I is much safer over processes disclosed in the prior art and therefore process of the present invention is novel, inventive and proves to be technically advanced process.

### DETAILED DESCRIPTION OF THE INVENTION

Disclosed herein is safe, scalable process providing innovative solutions and an equal balance between process and analytical parameters which work side-by-side to develop safe, scalable, efficient and cost-effective chemical processes for the preparation of 3-Fluoro-2-nitro Pyridine of formula I.

The preferred embodiments described herein details for illustrative purposes only and are by no means limiting and can be further enhanced by many variations. It is understood that various omissions and substitutions of equivalents are contemplated as circumstances may suggest or render expedient but are intended to cover the application or implementation without departing from the spirit or scope of the present invention.

With detailed reference to certain embodiments of the present invention, the example of these embodiments illustrates with structure and the formula enclosed. Although the present invention will be illustrated in conjunction with row illustrated embodiments, but it is to be understood that they are not intended to limit the invention to those embodiments.

It is contemplated that it contain all alternative forms, modification and equivalents that may be included in the scope of the invention as defined in the claims. The invention is not restricted to method described herein and material, but also includes any method similar or equivalent with method described herein and material that can be used for putting into practice the present invention and material.

Disclosed herein is an efficient process for the preparation of 3-Fluoro-2-nitro Pyridine of formula I comprising diazotization of compound 3-amino-2-nitropyridine of formula III with aqueous solution of sodium nitrite in presence of fluoroboric acid resulting into the formation of fluoroborate salt of formula II followed by its thermal decomposition to obtain the 3-Fluoro-2-nitro pyridine of formula I as depicted herein below as SCHEME A:

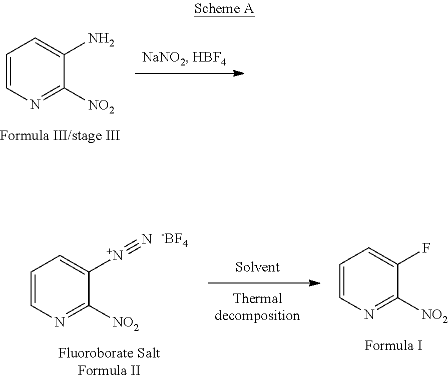

However; it is to be noted that inventors of the present invention have studied thermo chemistry of the reaction involved specifically transit of fluoroborate salt of Formula II into desired 3-Fluoro-2-nitro Pyridine of Formula I to ascertain the risk and hazards associated with the prior art processes discussed herein above and accordingly develop a safe and economical process at industrial scale.

More precisely, disclosed herein is an efficient process for the decomposition of the fluoroborate salt of formula II resulting into the formation of 3-Fluoro-2-nitro Pyridine of formula I comprising lot wise addition of fluoroborate salt of formula II in a high boiling solvent at about 85-95° C. and maintaining it till the completion of the reaction. Solvent is removed and desired product is obtained by distillation under the reduced pressure followed by crystallisation from low boiling solvent. Preferably solvent is high boiling such as toluene and the like.

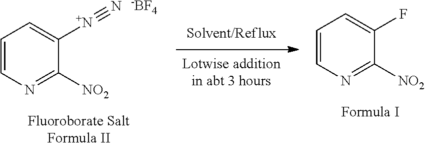

The invention describes in detail in connection with certain preferred and optional embodiments, so that various aspects thereof may be more fully understood and appreciated.

### Instrumental Parameters:

Inventors of the present invention have studied DSC and the hazards associated with prior art processes and also have evaluated the safety of the process of the present invention by performing ARC.

In one of the study, inventors of the present invention have performed DSC analysis for the conversion of compound of formula II into compound of formula I wherein the DSC is performed by taking only 0.867 g of fluoroborate shows 1^{st} exotherm onset at 97° C. with evolution of heat 135 J/g of sample, 2^{nd} exotherm onset at 179° C. with evolution of heat 72 J/g of sample and 3^{rd} Partial exotherm onset at 310° C. with evolution of heat 27 J/g.

Inventors of the present invention have performed DSC analysis for the conversion of compound of formula II i.e. fluoroborate salt into compound of formula I wherein the DSC is performed by taking only 5.6700 mg with the multiple exotherms with precise parameters comprising integral, normalised, peak, left limit and right limit as depicted in figure I herein below:

### BRIEF DESCRIPTION OF THE FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. I: Illustrates the DSC thermogram for the conversion of compound of formula II i.e. fluoroborate salt into compound of formula 1 the (Stage 4 to stage 5)

FIG. II: Illustrates the ARC analysis for the conversion of compound of formula II i.e. fluoroborate salt into compound of formula 1 the (Stage 4 to stage 5)

Liberation of this much thermal energy and multiple exotherms indicates warning alarm, thereby indicating the prior art process to be unsafe and responsible for low purity and yield.

Inventors of present invention based on their expertise and exhaustive R&D efforts provide herein a solution to the problem by adding compound of Formula II (i.e. Stage 4) into solvent in small portions which avoids exotherm and enables to maintain the adequate temperature profile which ensures completion of reaction avoiding generation of unsafe conditions viz foaming, high pressure, spillages and the likes thus making the process safe at industrial scale. Also the improved novel process gives substantially pure product in high yield.

The present invention can be understood by having experimentation including collection of negative data along with the example related to the present invention.

To collect the real data based on prior art references for the subject matter product, Inventors of the present invention have studied decomposition of 100 g fluoroborate salt of formula II in a single lot addition as disclosed in prior art in 1.5 lit toluene at 30-35° C. followed by slow heating to reach at refluxing, during which a lot of frothing was observed with sudden rise in temperature from 65° C. to 100° C. with gas evolution. The reaction is continued and brought to room temperature when toluene was decanted followed by it distillation under reduced pressure at 60-65° C. followed by degassing till the constant weight. About 65 grams degassed material yielded only 10 grams of pure 2-nitropyridine-3-amine of formula I, i.e. only 15% of degassed mass with the purity of 98%.

In another embodiment, with the intension of collecting the real data based on prior art process, inventors of the present invention have studied decomposition of 25 gm dry fluoroborate salt of formula II by heating it slowly to raise the temperature which resulted into Vigorous gas evolution & temperature increased from 45 to 120° C. and the whole material got charred.

In another general embodiment a solution of sodium nitrite in water is added drop wise to a stirred mixture of 2-nitropyridine-3-amine in 34% fluoroboric acid. During the addition temperature is maintained at −8° to about −2° C. Reaction mass is stirred at about −8° to −2° C. till the completion of the diazotization. Reaction mass is then filtered off to collect the fluoroborate solid washed with chilled 34% Fluoroboric acid followed by petroleum ether washing. Suck dry the product. Dry it under vacuum. Fluoroborate salt thus obtained is then charged in lots in a solvent maintained at about 85-95° C. in 4 hours. Heat the reaction mass to reach at reflux and maintained at least for 4 hours till the completion of the reaction. Reaction mass is cooled to RT and solvent is decanted followed by distillation of solvent under the reduced pressure and the mass so obtained is degassed and subjected for high vacuum distillation to collect the desired product. This is further followed by purification using low boiling solvent to get desired product as solid. Yield 30-35% with Purity 98.67.

In another specific embodiment a solution of sodium nitrite in water is added drop wise to a stirred mixture of 2-nitropyridine-3-amine in 34% fluoroboric acid. During the addition temperature is maintained at −8° to about −2° C. Reaction mass is stirred at about −8° to −2° C. till the completion of the diazotization. Reaction mass is then filtered off to collect the fluoroborate solid washed with chilled 34% Fluoroboric acid followed by petroleum ether washing. Suck dry the product. Dry it under vacuum. Fluoroborate salt thus obtained is then charged in lots in toluene maintained at about 85-95° C. in 4 hours. Heat the reaction mass to reach at about 105-110° C. and maintain the reflux at least for 4 hours till the completion of the reaction. Reaction mass is cooled to RT and toluene is decanted followed by distillation of toluene under the reduced pressure at about 60-65° C. and the mass so obtained is degassed and subjected for high vacuum distillation to collect the desired product. This is further followed by purification using low boiling solvent to get desired product as solid. HPLC Purity NLT 98% yield 30 to 35% obtained.

In another general embodiment of the invention the process as depicted in SCHEME-1, comprising decomposition of the fluoroborate compound of formula II is added in small lots in a high boiling solvent maintaining the temperature about 85-95° C. and heating/reflux is continued till the reaction gets completed. Solvent is distilled off from the reaction mass; residue so obtained is then taken for high vacuum distillation at temperature at about 60-65° C. to get the desired 3-Fluoro-2-nitro Pyridine as a major fraction which gets solidified yielding a yellow colored solid.3-Fluoro-2-nitro Pyridine thus obtained is further purified by crystallization using suitable low boiling solvent that can crystallize the desired compound of formula I.

High boiling solvent used in the said embodiment is selected from the group comprising solvents having high boiling points which shall solve the said purpose of decomposition of compound of formula II yielding compound of formula I. Preferable solvent is selected from the group comprising toluene, xylenes (o. m and p and the like).

Low boiling solvents used for the purpose of purification is selected from the group comprising. petroleum ethers, dialkyl ethers and the like.

In another specific embodiment a solution of sodium nitrite in water is added drop wise to a stirred mixture of 2-nitropyridine-3-amine in 34% fluoroboric acid. During addition the temperature is maintained at −8° to −2° C. Reaction mass is stirred-8° to −2° C. till the completion of the reaction. Reaction mass is then filtered off to collect the fluoroborate solid washed with chilled 500 ml 34% Fluoroboric acid followed by ether washing. Suck dry the product. Dry it under vacuum.

The invention is best understood by having the below given examples:

### Example I

A solution of sodium nitrite (502 gram) in water (960 mL) is added drop wise to a stirred mixture of 2-nitropyridine-3-amine (1000 grams) in 34% fluoroboric acid (2830 mL). During addition the temperature is maintained at −8° to −2° C. Reaction mass is stirred-8° to −2° C. till the completion of the reaction mass. Reaction mass is then filtered off to collect the fluoroborate solid washed with chilled 500 ml 34% Fluoroboric acid followed by ether washing. Suck dry the product. Dry it under vacuum. Entire Fluoroborate salt thus obtained is then charged in 30 lots in toluene maintaining the temperature at 85-95° C. in about 4 hours. Heat the reaction mass to reach at 105-110° C. and maintain the reflux at least for 4 hours till the completion of the reaction. After the completion of the reaction, reaction mass is cooled to RT and toluene is decanted followed by distillation of under the reduced pressure at 60-65° C. and the mass so obtained is degassed and subjected for high vacuum distillation to collect the desired product. This is further followed by purification using ether as solvent to get desired 3-Fluoro-2-nitro Pyridine as solid.

| All the results are tabulated | | |
|---|---|---|
| herein below: Particulars | Weight in grams | HPLC Purity |
| Degassed mass | 345 gm | 76.77% HPLC |
| 1^{st} cut | 12 gm | 74.42% HPLC |
| Main cut | 249 gm | 84.74% HPLC |
| Crystallized solid | 220 gm | 98.67% HPLC |

Wherein % of crystalline solid w.r.t. degassed mass is 63% against 15% obtained from that of negative experiment as per reported prior art.

As per the thermal data of reaction stage 5 (decomposition of stage 4 to stage 5) by ARC as depicted in Figure II, reveals thatmaximal temperature of synthesis reaction (Maximum temperature which can be reached in case adiabatic conditions) is 95° C.

ARC study reveals the following:
a. There is no thermal hazard when decomposition is performed by use offluoroborate salt of formula II is carried out in small lots.
b. No apparent side reaction expected in case of lot wise addition, on the contrary if added in single lot then vigorous boiling of toluene will happen charring the material and formation of impurities.
c. Addition in lots causes/indicates only a mild boiling.
d. No reaction accumulation is observed, as after every lot of stage-4 addition, enough digestion time is given.
e. Process comprising lot wise addition ensures that gas evolution stops completely before adding further lots,
f. Assessment of severity is low,
g. Decomposition is not critical.
h. Since the decomposition is low severe no major concern as per DSC data

All these parameters are supported by the analytical report as furnished herein below:

All these observation and study reveals that the process developed by the present inventors is safe in all aspect when the stage 5 decomposition involving transit of compound of formula II into I is conducted in lots as described herein before.

As per list of the hazards as depicted herein below indicates that decomposition of single lot of fluoroborate compound of formula II is at high risk of exotherms, associated with evolution of gas results in pressure buildup further the flue gas BF₃ is corrosive in nature hence its unsafe to practice it.

However, when decomposition of fluoroborate compound of formula II is done by the lot wise addition there is low risk and has the case in controlling the exotherm and gas evolution both . . . thereby provides a safe process for the preparation of 3-fluoro-2-nitro pyridine.

1. A process for the preparation of 3-fluoro-2-nitro pyridine of formula I 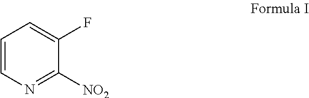
comprising:
a. Diazotization of 3-amino-2-nitropyridine of formula III with sodium nitrite in presence of fluoroboric acid as represented herein below to afford a compound of Formula II; 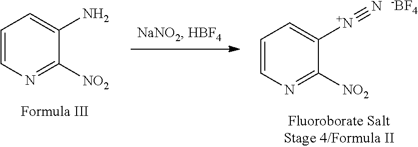
ii. isolation of the compound of formula II from the reaction mass of step I; 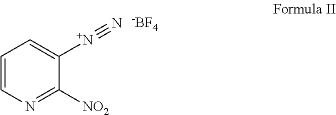
iii. decomposition of compound of formula II obtained in step ii comprising its lot wise addition in to a solvent resulting into formation of compound of formula I; 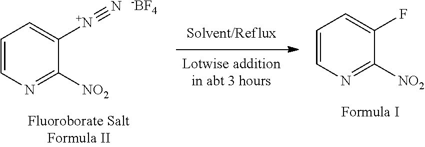
iv. maintaining refluxing till the completion of the reaction;
v. removal of solvent from the reaction mass followed by degassing the reaction mass;
vi. distillation under reduced pressure to obtain desired product 3-fluoro-2-nitro pyridine of formula I;
vii. purification of 3-fluoro-2-nitro pyridine of formula I obtained from stage vi using suitable solvent to obtain pure 3-fluoro-2-nitro pyridine with high purity 98.67%.

2. The process of claim 1, wherein temperature for diazotization is-8° C. to −2° C.

3. The process of claim 1, wherein acid used is fluoroboric acid.

4. The process of claim 1, wherein the temperature for the addition of compound of formula II is 85° C.-95° C.

5. The process of claim 1, wherein solvent is selected from group comprising toluene, m-xylene, o-xylene, p-xylene or mixture thereof.

6. The process of claim 4, wherein said solvent is toluene.

7. The process of claim 1, wherein solvent is selected from group comprising, ether, petroleum ether, hexane, heptanes or mixture thereof.

8. The process of claim 7, wherein said solvent is petroleum ether.
